(19) United States
(12) Patent Application Publication
(10) Pub. No.: US 2006/0222627 A1
(43) Pub. Date: Oct. 5, 2006

(54) Optimizing pharmacodynamics of therapeutic agents for treating vascular tissue

(76) Inventor: Andrew Carter, Portage, MI (US)

Correspondence Address: INTELLECTUAL PROPERTY / TECHNOLOGY LAW, PO BOX 14329, RESEARCH TRIANGLE PARK, NC, 27709, US

(21) Appl. No.: 11/096,241

(22) Filed: Mar. 30, 2005

(51) Int. Cl. A61K 38/20; A61K 31/7048; A61K 31/4745; A61K 31/553; A61K 31/551; A61K 31/496; A61K 31/4178; A61K 31/56; A61K 36/752

(52) U.S. Cl. 424/85.2; 514/171; 514/28; 514/29; 514/211.07; 514/456; 514/254.07; 514/27; 514/253.08; 514/355; 514/263.34; 514/397; 424/736; 514/479; 514/269; 514/220; 514/291

(57) ABSTRACT

An implant such as a stent is coated with a biodegradable or non-biodegradable polymer having therein an antiproliferative/immunosuppressive agent and a compound which reduces the rate of metabolism of the antiproliferative/immunosuppressive agent thereby inhibiting restenosis.

### FIELD OF THE INVENTION

The present invention relates to drugs and drug delivery systems for the prevention and treatment of vascular disease, and more particularly to drugs and drug delivery systems for the prevention and treatment of restenosis and neointimal hyperplasia.

### BACKGROUND OF THE INVENTION

Atherosclerosis, the major cause of ischemic heart disease, involves the production of stenotic lesions on the interior walls of coronary arteries which limit or obstruct coronary blood flow. One approach to treating an artery that has been constricted or occluded due to stenosis is percutaneous transluminal coronary angioplasty (PTCA) which is often followed by stent placement at the stenotic site. In this procedure, a balloon catheter is inserted and expanded in the constricted portion of the vessel for clearing the blockage. An increase in the use of this procedure is attributable to its relatively high success rate and its minimal invasiveness compared with coronary artery bypass surgery. However, PTCA is not without its limitations. Associated with PTCA is the abrupt closure of the treated vessel which may occur immediately after the procedure, and restenosis, or the renarrowing of the blood vessel, which occurs gradually following the procedure. About one-third of patients who undergo PTCA suffer from restenosis within about six months of the procedure. Restenosis is also a common problem in patients who have undergone saphenous vein bypass grafting.

While the exact mechanism of restenosis is not completely understood, it is generally known that multiple factors, including thrombosis, inflammation, growth factor and cytokine release, cell proliferation, cell migration and extracellular matrix synthesis contribute to the restenotic process. Upon expansion of an intracoronary balloon catheter during angioplasty, smooth muscle cells within the vessel wall become injured, initiating a thrombotic and inflammatory response. Cell-derived growth factors, such as platelet-derived growth factor, fibroblast growth factor, epidermal growth factor, thrombin, etc., are released from platelets, invading macrophages and/or leukocytes. Further, proliferative and migratory responses occur in medial smooth muscle cells. These cells undergo a change from the contractile phenotype to a synthetic phenotype characterized by only a few contractile filament bundles, extensive rough endoplasmic reticulum, Golgi and free ribosomes. Daughter cells migrate to the intimal layer of arterial smooth muscle and continue to proliferate and secrete significant amounts of extracellular matrix proteins. Proliferation, migration and extracellular matrix synthesis continue until the damaged endothelial layer is repaired at which time proliferation slows within the intima, usually within seven to fourteen days post-injury. The newly formed tissue is called neointima. The further vascular narrowing that occurs over the next three to six months is due primarily to negative or constrictive remodeling. Simultaneous with local proliferation and migration, inflammatory cells invade the site of vascular injury. Within three to seven days post-injury, inflammatory cells have migrated to the deeper layers of the vessel wall.

The most effective treatment currently known for preventing restenosis is the drug-eluting stent. Stents prevent negative remodeling but are associated with greater formation of neointima than balloon angioplasty. These stents are coated or impregnated with one or more therapeutic agents, which either reduce or prevent a hyperproliferative response at the site of implantation. Typically, the stent incorporates a biodegradable or nondegradable, polymer-based matrix to provide controlled release of therapeutic agents within the blood vessel. The release mechanism of the drug from the polymeric material depends on the nature of the polymeric material and the drug itself. Release of the drug occurs by diffusion from or degradation of the polymeric material. Degradation of the polymeric material occurs through hydrolysis, which erodes the polymer into the fluid and hence releases the drug into the fluid as well.

An important consideration in using drug-eluting stents is the release rate of the drug. It is desirable that an effective therapeutic amount of the drug be released from the stent for the longest period of time possible. Burst release, a high release rate immediately following implantation, is undesirable as it wastes the limited supply of the drug by releasing several times the effective amount required and shortens the duration of the release period. Additionally, this may be harmful to the patient where the agent or its metabolites are toxic at higher doses.

Various types of antiproliferative and immunosuppressive agents have been employed with drug eluting stents to prevent restenosis. These include sirolimus, everolimus, ABT-578, FK 506, cyclosporine, mycophenolic acid (and its prodrug form as mycophenolate mofetil), and pimicrolimus. These agents are bacterial (sirolimus, FK 506) or fungal (cyclosporine A) metabolites that suppress lymphocyte function and cellular proliferation. Because of their toxicities, these agents cannot be used at maximally immunosuppressive doses. Accordingly, there is a need for safer versions of these agents as well as analogues thereof with higher immunosuppressive efficacy.

The other significant issue that complicates the delivery of relatively high dosages of these agents is their relatively narrow therapeutic application. While certain combinations of these agents, in some circumstances, have a broader application, their cumulative toxicity restricts most of these agents to use as a monotherapy in intravascular delivery applications. Still yet, in certain combinations their therapeutic effects may be contraindicated as one agent may counteract or thwart the other's intended effects. However, in other combinations, the toxicity of one or more of these agents in combination with another agent is reduced while their effectiveness is increased. For example, sirolimus at highly effective therapeutic doses is highly toxic but in combination with cyclosporine A, its therapeutic effect is significantly increased when compared with monotherapy, and as such, a lower dosage of the combination can be used which is less toxic or non-toxic.

The antiproliferative effects of these compounds are dependent, in part, on dose, arterial tissue concentration, and the residence time of the drug in the arterial wall. Drug loading on the strut surface of a stent is limited by surface area and polymer characteristics such as thickness of the coating. For polymeric stent surface-coated drug delivery systems, the maximal drug load is severely constrained by the physical properties of the material. Furthermore, drug elution from a solid matrix strut surface coating depends on the physical properties of the compound and of the polymer generally within principles of concentration dependent drug diffusion. The arterial disposition of the compound may be influenced by the physical properties of the drug, active cellular mechanisms of drug-uptake, vascular morphology, redistribution to systemic circulation, and metabolism or degradation in the vessel wall.

It is known that cytochrome P450 (CYP) is responsible for metabolism of some antiproliferative and immunosuppressive compounds. Experimental studies have documented the expression of CYP in the endothelial and vascular smooth muscle cells of the arterial wall. In fact, it has been demonstrated that endothelial cells contain several heme-containing enzymes including CYP, nitric oxide synthase, and prostacyclin synthase. See Pfister et al., Rabbit aorta converts 15-HPETE to trihydroxyeicosatrienoic acids: potential role of cytochrome P450, Arch Biochem Biophys, 2003 Dec. 1;420(1):142-52.

CYP 3A4, which participates in the formation of nitric oxide from the compound isosorbide dinitrate and is present in the endothelium of human coronary arteries, has been found to be responsible for hepatic metabolism of compounds including sirolimus, ABT-578, everolimus, FK506 and pimicrolimus and their analogs. For example, it is known that sirolimus is metabolized via the CYP 3A4 to the inactive metabolites 41-hydroxy and 39-demethyl sirolimus which do not exhibit any antiproliferative or immunosuppressive properties.

Several compounds are known to modulate CYP substrate. These compounds can produce non-specific or specific effects on CYP substrate. Clotrimazole is an example of a non-specific CYP inhibitor while ebastine and terfenadone specifically inhibit CYP 2J2. Compounds that block or reduce the CYP 3A4 are known to prolong the half-life of sirolimus and its analogs. For example, ketoconazole, oleandomycin and gestodene are selective inhibitors of CYP3A4 that inhibit metabolism of FK 506 and sirolimus.

It would be advantageous to provide a composition to modify or modulate CYP in the arterial wall in order to prevent arterial wall biotransformation of immunosuppressive-antiproliferative compounds and, thus, increase the arterial tissue concentration and/or tissue half-life of a stent-based agent.

### SUMMARY OF THE INVENTION

The present invention comprises a combination of two compounds wherein a first compound acts to suppress lymphocyte function and cellular proliferation and a second compound inhibits the formation of and/or activity of an enzyme involved in the metabolism of the first compound. The first compound, which is an antiproliferative-immunosuppressive agent, is useful in inhibiting restenosis. The second compound maintains the activity of the first compound by either inhibiting an enzyme involved in metabolizing the first compound or inhibiting the formation of compounds which are involved in the metabolism of the first compound.

An aspect of the invention is the local inhibition of CYP 3A4 in the arterial wall thereby prolonging the tissue half-life, reducing the formation of degradants, and inhibiting other mechanisms which results in metabolism of compounds such as sirolimus.

Another aspect of the invention is the preservation of compounds in the vessel wall via inhibition of CYP which results in reducing the presence of metabolites or degradants associated with idiosyncratic or other adverse drug reactions.

Still another aspect of the invention provides specific CYP modulators that alter the expression of the CYP and therefore the metabolism of a particular agent such as an immunosuppressive and/or antiproliferative compound.

Still yet another aspect of the invention is providing such CYP modulators to enable the use of antiproliferative-immunosuppressive agents at dosages having lower toxicity levels and/or which improve the efficacy of the antiproliferative agents by altering the tissue or cellular pharmacodynamics of the compound.

Another aspect of the invention is a CYP modulator and antiproliferative agent combination used in the context of PTCA and/or stent placement within a vessel, e.g., at a target site within the wall of the artery being treated.

Another aspect of the present invention is the combination of parenteral or local agents that modify CYP3A4 in order to increase the arterial tissue concentration and/or tissue half-life of stent-based, antiproliferative-immunosuppressive agents.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawing. It is emphasized that, according to common practice, the various features of the drawing are not to-scale. On the contrary, the dimensions of the various features are arbitrarily expanded or reduced for clarity.

FIG. 1 is a schematic illustration of a stent coated with an exemplary CYP inhibitor, e.g., ketoconazole, and an exemplary suppressor of cell proliferation, e.g. sirolimus. Biochemical interactions are also schematically shown. For example, ketoconazole inhibits CYP 3A4 and thereby inhibits the metabolism of sirolimus to 39-demethyl sirolimus. Sirolimus inhibits cytokines and modulates cyclin dependent kinases involved in cellular proliferation.

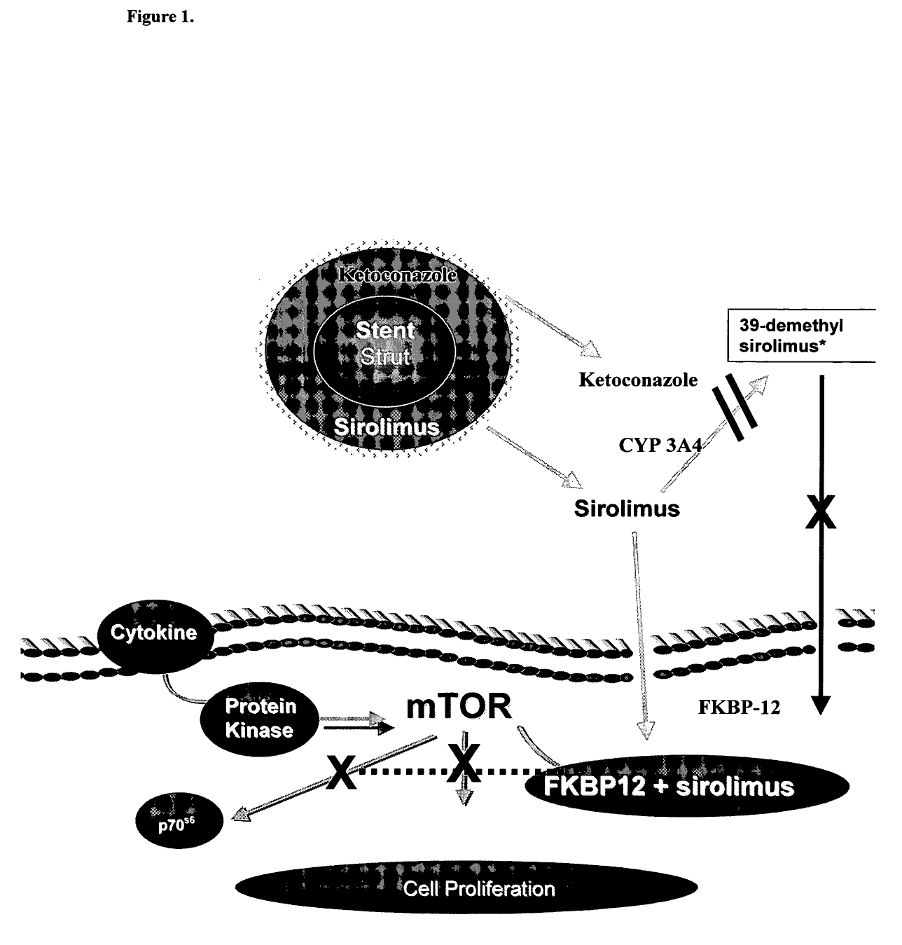

### DETAILED DESCRIPTION OF THE INVENTION

Before the present invention is described, it is to be understood that this invention is not limited to particular drug or agent combinations described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. It must be noted that as used herein and in the appended claims, the singular forms “a”, “an”, and “the” include plural referents unless the context clearly dictates otherwise. Thus, for example, reference to “agent” may include a plurality of such agents and reference to “the implant” includes reference to one or more implants and equivalents thereof known to those skilled in the art, and so forth.

All publications mentioned herein are incorporated herein by reference to disclose and describe the methods and/or materials in connection with which the publications are cited. It is understood that the present disclosure supercedes any disclosure of an incorporated publication to the extent there is a contradiction. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Although any methods and materials similar or equivalent to those described herein can be used in the practice or testing of the present invention, the preferred methods and materials are now described.

### Invention in General

The present invention describes methods to modify arterial biotransformation of drugs to metabolites without known biological or potentially toxic effects by altering metabolic pathways of certain compounds in the vessel wall. The methods and compositions of the invention enable the application of stent-based delivery of antiproliferative-immunosuppressive drugs at lower doses, enhance efficacy via sustained arterial tissue concentrations of the drug, and reduce the likelihood for idiosyncratic or hypersensitivity reactions.

The present invention combines the parenteral or local delivery of agents which inhibit or induce CYP activity or production in the arterial wall and thereby increase the arterial tissue concentration of and/or the tissue half-life of a locally delivered antiproliferative-immunosuppressive drug, as well as alter the production of metabolites in the arterial wall. The metabolic activity of CYP in the arterial wall is inhibited or induced by systemic or local administration of the CYP inhibitor or inducer, respectively.

In one embodiment, the CYP inhibitor/inducer is delivered locally to the artery, e.g., via a balloon catheter, a stent or other medical implant. The CYP inhibitor/inducer may be coated directly to the entire exposed surface of the stent or a portion thereof, which may have a metal or non-polymeric structure, or be embedded within a biodegradable or non-biodegradable polymer matrix applied to the stent body. In either configuration, the CYP inhibitor/inducer may be combined with one or more antiproliferative and/or immunosuppressive compounds in equimolar or varying concentrations to achieve the desired biological effect, i.e., enhance the half-life of the antiproliferative and/or immunosuppressive agent and thereby inhibit restenosis.

Additionally, the coating or embedding of the CYP inhibitor or inducer may be provided in a manner to allow for any sequence of drug delivery. For example, the coating or embedding of the drugs may be done to provide for an initial delivery of the CYP inhibitor/inducer followed by delivery of the antiproliferative and/or immunosuppressive compound. Alternatively, the drugs may be coated or embedded in a manner which provides for the simultaneous delivery of the CYP inhibitor/inducer and the antiproliferative/immunosuppressive compound. In another embodiment, the drugs may be coated or embedded in a manner which provides for the initial delivery of the antiproliferative/immunosuppressive compound followed by delivery of the CYP inhibitor/inducer.

Use of a CYP inhibitor in the arterial wall may facilitate other mechanisms of drug metabolism, thereby reducing the presence of metabolites or degradants that may be associated with idiosyncratic or other adverse drug reactions. In one variation, the CYP inhibitor used with the methods of the present invention is a CYP 450 3A4 inhibitor. In one embodiment, the CYP 450 3A4 inhibitor is ketoconazole, and the antiproliferative/immunosuppressive compound is sirolimus. The dose of ketoconazole may range from about 0.0001 to about 100 mg per millimeter of stent length or square millimeter of the medical implant surface, and the dose of sirolimus may range from about 0.0001 to about 100 mg per millimeter of stent length or square millimeter of the medical implant surface.

Drugs or compounds that induce or increase CYP substrate can promote the metabolism of other drugs or compounds that depend on this metabolic pathway. Thus, a CYP inducer would increase the arterial metabolism of several drugs resulting in a reduced tissue half-life. In one variation, the CYP 450 inducer is rifampin and the immunosuppressive compound is ABT-578, a tetrazole ring analog of sirolimus with a prolonged tissue half-life. The dose of ABT-578 may range from about 0.0001 to about 1100 mg per millimeter of stent length or square millimeter of the medical implant surface. The dose of rifampin may range from about 0.0001 to about 100 mg per millimeter of stent length or square millimeter of the medical implant surface.

Other commonly known CYP inhibitors that might be used in combination with sirolimus or an analog thereof include but are not limited to amiodarone, azithromycin, cimetidine, ciprofloxacin, imidazole antifungals, clarithromycin, clotrimazole, calcium channel blockers such as nifedipine, diltiazem and verapamil, delaviridine, diethyldithiocarbamate, erythromycin, fluconazole, fluvoxamine, gestodene, grapefruit juice, indinavir, interleukin-10, itraconazole, mibefradil, mifepristone, nefazodone, nelfinavir, naringen, norfloxacin, norfluoxetine, ritonavir, saquinavir, and troleandomycin.

Other commonly known CYP inducers that might be used in combination with sirolimus or an analog include but are not limited to the herbal medicine St. John's Wort, barbiturates, carbamazepine, efavirenz, glucocorticoids, modafinil, nevirapine, phenobarbital, phenyloin, pioglitazone, and troglitazone.

Other immunosuppressive compounds with antiproliferative properties include ABT-578, biolimus A9, everolimus, FK506, pimicrolimus, methotrexate, and steroids.

Those skilled in the art will understand that still other immunosuppressive compounds, CYP inhibitors and CYP inducers are known and will be developed. The present invention intends to encompass such by teaching the combinations provided herein which increase the half-life or useful active life of any given immunosuppressive compound and thereby inhibit restenosis. Accordingly, the examples described are not intended to limit applicability of this invention to other combinations of CYP inhibitors/inducers and antiproliferative/immunosuppressive compounds for the prevention of restenosis.

As mentioned above, the compositions of the present invention may be delivered with a combination of delivery routes. For example, the immunosuppressive compound may be delivered locally as a coating on a medical implant, e.g., a stent, while the CYP modulator may be delivered systemically, e.g., through oral and/or parenteral delivery. The oral or ingestible form of the modulator may be a liquid or solid (pill form). Parenteral administration may be via intravenous, intra-arterial, intra-articular, intracardiac, subcutaneous, intradermal, intraspinal or epidural, or intramuscular injection. Oral administration is advantageous in that it can be done conveniently by the patient, however, but is clearly not suitable for continuous administration. Parenteral administration, on the other hand, is not as convenient for the patient and may require the assistance of a medical professional; however, it can be administered continuously or for periods of continuous delivery.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A composition comprising:
a CYP modulator; and
an antiproliferative/immunosuppressive agent;
wherein the weight ratio of the CYP modulator to the antiproliferative/immunosuppressive agent is in a range of from about 1:100000 to about 100000:1.

2. The composition of claim 1, further comprising:
a biodegradable or non-biodegradable polymer.

3. The composition of claim 1, wherein the CYP modulator is a CYP inhibitor.

4. The composition of claim 3, wherein the CYP 450 inhibitor is diltiazem.

5. The composition of claim 3, wherein the CYP inhibitor is a selective CYP 3A4 inhibitor.

6. The composition of claim 5, wherein the selective CYP 3A4 inhibitor is ketoconazole.

7. The composition of claim 1, wherein the antiproliferative/immunosuppressive agent is sirolimus or an analog thereof.

8. The composition of claim 3, wherein the CYP inhibitor is selected from the group consisting of amiodarone, azithromycin, cimetidine, ciprofloxacin, imidazole antifungals, clarithromycin, clotrimazole, calcium channel blockers, delaviridine, diethyl-dithiocarbamate, erythromycin, fluconazole, fluvoxamine, gestodene, grapefruit juice, imidazole antifungals, indinavir, interleukin-10, itraconazole, ketoconazole, mibefradil, mifepristone, nefazodone, nelfinavir, naringen, norfloxacin, norfluoxetine, ritonavir, saquinavir, and troleandomycin.

9. The composition of claim 1, wherein the CYP modulator is a CYP inducer.

10. The composition of claim 9, wherein the CYP inducer is a CYP 3A4 inducer.

11. The composition of claim 10, wherein the CYP 3A4 inducer is rifampin and the antiproliferative/immunosuppressive compound is ABT-578.

12. The composition of claim 9, wherein the CYP inducer is selected from the group consisting of St. John's Wort, barbiturates, carbamazepine, efavirenz, glucocorticoids, modafinil, nevirapine, phenobarbital, phenyloin, pioglitazone and troglitazone.

13. The composition of claim 1, wherein the antiproliferative/immunosuppressive agent is selected from the group consisting of ABT-578, biolimus A9, everolimus, FK506, pimicrolimus, methotrexate and steroids.

14. A medical implant, comprising:
a structure having the composition of claim 1 coated thereon or embedded therein.

15. The medical implant of claim 14, wherein the structure is a stent.

16. The medical implant of claim 14, further comprising:
a biodegradable or non-biodegradable polymer.

17. The medical implant of claim 16, wherein the composition is applied to the structure in a manner that allows for the elution of the CYP modulator independently of the antiproliferative/immunosuppressive agent.

18. The medical implant of claim 16, wherein the CYP modulator is eluted prior to elution of the antiproliferative and/or immunosuppressive agent.

19. The medical implant of claim 16, wherein the CYP modulator is eluted after elution of the antiproliferative and/or immunosuppressive agent.

20. The medical implant of claim 16, wherein the composition is applied to the structure in a manner that allows for the elution of the CYP modulator simultaneously with the antiproliferative/immunosuppressive agent.

21. The medical implant of claim 16, wherein the composition is applied to the structure in a manner that allows for the elution of the CYP modulator intermittently with the antiproliferative and/or immunosuppressive agent.

22. The medical implant of claim 16, wherein the CYP modulator is a CYP inhibitor.

23. The medical implant of claim 22, wherein the CYP inhibitor is a CYP 3A4 inhibitor.

24. The medical implant of claim 23, wherein the CYP 3A4 inhibitor is ketoconazole.

25. The medical implant of claim 24, wherein the dose of ketoconazole ranges from about 0.0001 to about 100 mg per square millimeter of the surface of the medical implant.

26. The medical implant of claim 16, wherein the antiproliferative/immunosuppressive agent is sirolimus.

27. The medical implant of claim 26, wherein the sirolimus is provided in an amount in the range from about 0.0001 to about 100 mg per square millimeter of the surface of the medical implant.

28. A method of modulating CYP function in order to alter biotransformation of compounds in a vessel wall of a subject, the method comprising:
administering at a target site on a vessel wall an effective amount of an antiproliferative/immunosuppressive agent; and
administering to the subject an effective amount of a CYP modulator, wherein the weight ratio of the CYP inducer to the antiproliferative/immunosuppressive agent is about 1:100000 to about 100000:1; and
altering the arterial tissue concentration of the antiproliferative/immunosuppressive agent at the target site.

29. The method of claim 28, wherein the CYP modulator is a CYP inducer and the concentration of the antiproliferative/immunosuppressive agent at the target site is increased.

30. The method of claim 28, wherein the CYP modulator is a CYP inhibitor and the concentration of the antiproliferative/immunosuppressive agent at the target site is decreased.

31. The method of claim 28, wherein the CYP modulator is administered at the target site in the artery.

32. The method of claim 28, wherein the CYP modulator is administered systemically.

33. The method of claim 32, wherein the CYP modulator is administered orally.

34. The method of claim 32, wherein the CYP modulator is administered parenterally.

35. The method of claim 28, wherein the CYP modulator and the antiproliferative/immunosuppressive agent are administered independently.

36. The method of claim 35, wherein the CYP modulator is administered prior to administration of the antiproliferative and/or immunosuppressive agent.

37. The method of claim 35, wherein the CYP modulator is administered after administration of the antiproliferative and/or immunosuppressive agent.

38. The method of claim 35, wherein the CYP modulator and the antiproliferative/immunosuppressive agent are administered intermittently.

39. The method of claim 28, wherein the CYP modulator and the antiproliferative/immunosuppressive agent are administered simultaneously.

40. The method of claim 28, further comprising implanting a structure at the target site wherein the antiproliferative/immunosuppressive agent is coated on or embedded within the structure.

41. The method of claim 40, wherein the CYP modulator is coated on or embedded within the structure.

42. The method of claim 40, wherein the CYP modulator is configured for ingestion.

43. The method of claim 40, wherein the CYP modulator is configured for injection.
